(19) United States
(12) Patent Application Publication
(10) Pub. No.: US 2021/0008011 A1
(43) Pub. Date: Jan. 14, 2021

(54) METHODS FOR TREATING INFLAMMATORY DISEASES

(71) Applicants: Charles W. STRATTON, Nashville, TN (US); Nicole M. SCHULTEK, Wake Forest, NC (US)

(72) Inventors: Charles W. STRATTON, Nashville, TN (US); Nicole M. SCHULTEK, Wake Forest, NC (US)

(21) Appl. No.: 16/923,729

(22) Filed: Jul. 8, 2020

Related U.S. Application Data
(60) Provisional application No. 62/871,484, filed on Jul. 8, 2019.

(51) Int. Cl. A61K 31/145; A61K 31/047; A61K 31/438; A61K 31/65; A61P 29/00; A61K 31/7052; A61K 31/7034; A61K 31/7036; A61K 31/7048; A61K 31/573; A61K 31/58; A61K 31/519; A61K 38/13; A61K 31/436; A61K 31/42; A61K 31/5377; A61K 39/395; A61K 38/20

(52) U.S. Cl. CPC A61K 31/145; A61K 38/2006; A61K 31/438; A61K 31/65; A61P 29/00; A61K 31/7052; A61K 31/7034; A61K 31/7036; A61K 31/7048; A61K 31/573; A61K 31/58; A61K 31/519; A61K 38/13; A61K 31/436; A61K 31/42; A61K 31/5377; A61K 39/3955; A61K 39/39558; A61K 31/047

(57) ABSTRACT

The present invention relates to treatment of inflammatory diseases with agents that target different phases of the life cycle of Chlamydia spp. In particular, the invention features the use of a combination of one or more of taurine, dimethylglycine, and trimethylglycine with one or more of a rifamycin antibiotic, a macrolide antibiotic, and a tetracycline antibiotic for treating inflammatory diseases resulting from Chlamydial infection.

### BACKGROUND OF THE INVENTION

Chlamydiae are obligate intracellular microorganisms which parasitize eukaryotic cells and are ubiquitous throughout the animal kingdom. Members of the genus Chlamydia have a unique triphasic developmental cycle having distinct morphological and functional forms; these are the infectious elementary body, the replicating reticulate body, and the non-replicating cryptic body. The chlamydial life cycle alternates between: (a) an extracellular life form that is an infectious, metabolically-inactive form known as the elementary body (EB); and (b) intracellular life forms, of which two are currently recognized, a metabolically-active, replicating organism known as the reticulate body (RB) and a persistent, non-replicating organism known as the cryptic body. EBs are small (300-400 nm) infectious, spore-like forms which have recently been recognized as being metabolically active in the acellular milieu, non-replicating, and found most often in the acellular milieu. EBs are resistant to a variety of physical insults such as enzyme degradation, sonication and osmotic pressure. This physical stability is thought to be a result of extensive disulfide cross-linking of the cysteine-rich major outer membrane protein (MOMP). Under oxidizing conditions in the acellular milieu of the host, the outer membrane of EBs is relatively impermeable as well as resistant to inactivation. EBs are thus well suited to survive long enough outside of their hosts to be transmitted to a new host in the form of a droplet nuclei or a fomite.

Infection by members of the genus Chlamydia induces a significant inflammatory response at the cellular level. Yet, clinically, the initial infection is frequently varied in symptomatology and may even be asymptomatic. Once fully established, Chlamydia spp. are difficult to eradicate, with frequent relapse following antibiotic therapy. Evidence also indicates that Chlamydia spp. may become dormant and are then shed in quantities too few to reliably detect by cell culture. The current therapy for suspected/confirmed chlamydial infection is with a short course (e.g., 2-3 weeks) of a single antibiotic, such as macrolide, tetracyline, and fluoroquinolone, to which Chlamydia spp. is susceptible in vitro. However, despite this demonstration of in vitro susceptibility, chlamydial infections may relapse following treatment with these antibiotics. In vitro studies on the persistence of Chlamydiae despite specific and appropriate antibiotic therapy have suggested that the presence of antibiotics promotes the formation of an intracellular, non-replicative cryptic state (Beatty et al., Microbiol. Rev. 58:686-699 (1994)), typically referred to as the latent or cryptic body. This change can be thought of as a stringent response and is seen also with nutrient starvation and exposure to γ-interferon. Removal of the stressful influence allows the organism to resume replication. Thus, in this way, the organism can escape the antibiotic therapy that are presently used in clinical practice. In view of the chronic and persistent nature of chlamydial infections, and the ability of chlamydial EBs to escape antibiotic therapy, there is a need for antichlamydial therapy which totally eradicates this pathogen, including the elementary body phase, the cryptic phase and/or the replicating phase, thereby preventing the long-term sequelae of such chronic infections.

### SUMMARY OF THE INVENTION

Featured herein are methods for treating chronic inflammatory diseases resulting from chlamydial infection by using a combination of agents that target each of the different phases of chlamydial life cycle. In one embodiment, the invention features use of a combination of one or more of taurine, dimethylglycine, and trimethylglycine with one or more of a rifamycin antibiotic, a macrolide antibiotic, and a tetracycline antibiotic for treating inflammatory diseases that result from chlamydial infection.

A first aspect features a method of treating a chronic inflammatory disease (e.g., an inflammatory disease resulting from chlamydial infection) in a subject (e.g., a human) in need thereof by administering to the subject at least two agents, wherein each agent is effective against a different phase of chlamydial life cycle and is selected from the following groups: (a) agent effective against elementary body (EB) phase of chlamydial life cycle, such as one or more of taurine, dimethylglycine, and trimethylglycine; and (b) agent effective against cryptic phase and/or replicating phase of chlamydial life cycle, such as one or more of a rifamycin antibiotic, a macrolide antibiotic, and a tetracycline antibiotic, and wherein one or more agents from group (a) are administered within 24 hours (e.g., within 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5.5 hours, 5 hours, 4.5 hours, 4 hours, 3.5 hours, 3 hours, 2.5 hours, 2 hours, 1.5 hours, 1 hour, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, 1 minute, or less) of administering one or more agents from group (b).

In some embodiments of the first aspect, the method reduces or eliminates chlamydial infection in the subject. In some embodiments of the first aspect, the method further includes a test, wherein the test detects EB phase of chlamydial life cycle, replicating phase of chlamydial life cycle, and cryptic phase of chlamydial life cycle in a biological material (e.g., bodily secretion, bodily fluid, and/or tissue) from the subject, and the agents are administered to the subject until the biological material from the subject is negative for Chlamydia according to the test. In further embodiments, the test is an assay for detection of chlamydial nucleic acid or protein, and includes polymerase chain reaction, enzyme-linked immunosorbent assay (ELISA), radioimmunoassay, and/or immunohistology.

In some embodiments of the first aspect, the method of treatment reduces one or more symptoms of the inflammatory disease in the subject.

In some embodiments of the first aspect, the method of treatment further includes administering to the subject one or more immunosuppressive drugs, such as one or more of a corticosteroid (e.g., prednisone, budesonide, or prednisolone), a janus kinase inhibitor (e.g., tofacitinib), a calcineurin inhibitor (e.g., cyclosporine or tacrolimus), an mTOR inhibitor (e.g., sirolimus or everolimus), an IMDH inhibitor (e.g., azathioprine, leflunomide, or mycophenolate), a biologic (e.g., abatacept, adalimumab, anakinra, certolizumab, etanercept, golimumab, infliximab, ixekizumab, natalizumab, rituximab, secukinumab, tocilizumab, ustekinumab, or vedolizumab), or a monoclonal antibody (e.g., basiliximab or daclizumab).

A second aspect features a combination of at least two agents for use in treating a chronic inflammatory disease (e.g., an inflammatory disease resulting from chlamydial infection) in a subject (e.g., a human) in need thereof, wherein each agent is effective against a different phase of chlamydial life cycle and is selected from the following groups: (a) agent effective against elementary body (EB) phase of chlamydial life cycle, such as one or more of taurine, dimethylglycine, and trimethylglycine; and (b) agent effective against cryptic phase and/or replicating phase of chlamydial life cycle, such as one or more of a rifamycin antibiotic, a macrolide antibiotic, and a tetracycline antibiotic, and wherein one or more agents from group (a) are formulated for administration within 24 hours (e.g., within 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5.5 hours, 5 hours, 4.5 hours, 4 hours, 3.5 hours, 3 hours, 2.5 hours, 2 hours, 1.5 hours, 1 hour, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, 1 minute, or less) of administration of one or more agents from group (b).

In some embodiments of the second aspect, the combination of agents reduces or eliminates chlamydial infection in the subject. In additional embodiments of the second aspect, the combination of agents reduces one or more symptoms of the inflammatory disease in the subject.

In some embodiments of the second aspect, the combination further includes one or more immunosuppressive drugs, such as one or more of a corticosteroid (e.g., prednisone, budesonide, or prednisolone), a janus kinase inhibitor (e.g., tofacitinib), a calcineurin inhibitor (e.g., cyclosporine or tacrolimus), an mTOR inhibitor (e.g., sirolimus or everolimus), an IMDH inhibitor (e.g., azathioprine, leflunomide, or mycophenolate), a biologic (e.g., abatacept, adalimumab, anakinra, certolizumab, etanercept, golimumab, infliximab, ixekizumab, natalizumab, rituximab, secukinumab, tocilizumab, ustekinumab, or vedolizumab), or a monoclonal antibody (e.g., basiliximab or daclizumab).

In some embodiments of the second aspect, the combination is formulated as a pharmaceutical composition. In additional embodiments, the pharmaceutical composition further includes a pharmaceutically acceptable carrier, excipient, or vehicle.

In some embodiments of any of the above aspects, the one or more agents from group (a) include taurine; a combination of taurine and dimethylglycine; or a combination of taurine, dimethylglycine and trimethylglycine. In some embodiments, the taurine analog is N-chlorotaurine (NCT) (e.g., a 1% (w/v) aqueous solution of NCT). The skilled artisan would recognize that taurine has alternative names known the art (e.g., 2-aminoethanesulfonic acid, 107-35-7, L-Taurine, tauphon, Ethanesulfonic acid, 2-amino-Ethanesulfonic acid, 2-Aminoethylsulfonic acid, O-Due, 2-Sulfoethylamine, and taufon) and refer to the same chemical compound.

In some embodiments of any of the above aspects, the one or more agents from group (b) include a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline).

In some embodiments of any of the aforementioned aspects, the inflammatory disease is a respiratory disease (e.g., a respiratory condition involving inflammation or infection of a respiratory mucosa; a respiratory condition involving inflammation or infection of an underlying muscle of the respiratory tract; cystic fibrosis; pneumonia; asthma; bronchitis; sinusitis or rhinosinusitis; infection of a sinus; chronic obstructive airway disease; emphysema; chronic bronchitis; or bronchiectasis), a neurodegenerative disease (e.g., Alzheimer's disease, Parkinson's disease, Prion disease, motor neuron diseases, Huntington's disease, spinocerebellar ataxia, or spinal muscular atrophy), a musculoskeletal disease (e.g., arthritis, tendinitis, carpal tunnel syndrome, or fibromyalgia), or a cardiovascular disease (e.g., atherosclerosis, coronary artery diseases, stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, or venous thrombosis).

### Definitions

As used herein, the term “administering” refers to the act of providing or giving a subject a combination of agents, such as a combination of anti-chlamydial agents (e.g., a combination of: (a) at least one agent effective against (e.g., that effectively reduces or eliminates) the EB phase of chlamydial life cycle (e.g., one or more of taurine, dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic) and/or immunosuppressive drugs (e.g., one or more one or more of a corticosteroid, a janus kinase inhibitor, a calcineurin inhibitor, an mTOR inhibitor, an IMDH inhibitor, a biologic, or a monoclonal antibody), by any effective route (e.g., orally). Exemplary routes of administration are described herein below.

As used herein, the term “anti-chlamydial agent” refers to agents (e.g., therapeutic agents) that are effective against different phases of chlamydial life cycle (e.g., the EB phase, cryptic phase, and replicating phase of chlamydial life cycle). Anti-chlamydial agents include: (a) agents effective against the EB phase of chlamydial life cycle (e.g., one or more of taurine, dimethylglycine, and trimethylglycine); and (b) agents effective against the cryptic phase and/or replicating phase of chlamydial life cycle (e.g., one or more of a rifamycin antibiotic, a macrolide antibiotic, and a tetracycline antibiotic).

As used herein, the term “agents effective against a phase of chlamydial life cycle” refers to agents (e.g., therapeutic agents) that effectively reduces or eliminates that phase of chlamydial life cycle. For example, “agents effective against the EB phase of chlamydial life cycle” refers to agents that effectively reduces or eliminates that phase of chlamydial life cycle (e.g., one or more of taurine, dimethylglycine, and trimethylglycine). Alternatively, agents effective against the cryptic phase and/or replicating phase of chlamydial life cycle refers to agents that effectively reduces or eliminates the cryptic phase and/or replicating phase of chlamydial life cycle (e.g., one or more of a rifamycin antibiotic, a macrolide antibiotic, and a tetracycline antibiotic).

The term “effective amount” or “therapeutically effective amount,” as used herein, means an amount of an agent (e.g., an anti-chlamydial agent), that, when administered to a subject (e.g., a human subject or an animal model) in need of such treatment, is sufficient to effect treatment, as defined herein. For example, a combination of agents (e.g., an anti-chlamydial agent) in effective amount or therapeutically effective amount, is sufficient to reduce or eliminate chlamydial infection, reduce or eliminate the different phases of chlamydial life cycle (e.g., EB phase of chlamydial life cycle, cryptic phase of chlamydial life cycle, and replicating phase of chlamydial life cycle), and treat inflammatory diseases that are associated with, caused by or result from chlamydial infection.

The terms “treat”, “treating”, and “treatment” refer to any treatment of an inflammatory disease in a subject, such as a mammal, particularly a human, by the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine, dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) and include: reducing inflammation (e.g., reduce release of inflammatory cytokines (e.g., TNF-α, IFNγ, IL-1, IL-6, IL-1β, IL-12, IL-18, etc.), and/or reducing one or more symptoms of the inflammatory disease.

As used herein, the terms “increase,” “increasing,” “induce” or “inducing” and “decrease,” “decreasing,” “reduce” or “reducing” refer to modulating resulting in, respectively, greater or lesser amounts, of function, expression level, occurrence, or activity of a metric relative to a reference. For example, subsequent to administration of the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine, dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)), the different phases of chlamydial life cycle in a subject (e.g., in a biological material of the subject) may reduce or decrease by at least 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)) relative to the different phases of chlamydial life cycle prior to administration of the combination of agents. Generally, the metric is measured subsequent to administration at a time that the administration has had the recited effect, e.g., at least 3 hours, 6 hours, 12 hours, 18 hours, 1 day, 2 days, 3 days, 4 days, 5 days, 6 days, 7 days, 1 week, 2 weeks, 3 weeks, 4 weeks, 5 weeks, 6 weeks, 7 weeks, 8 weeks, 3 months, 4 months, 5 months, or 6 months, after a treatment regimen has begun. The term “reducing” is used interchangeably with the term “decreasing” herein. The term “increasing” is used interchangeably with the term “inducing” herein.

The terms “comprising” and “including” as used herein, are used in their open, non-limiting sense.

For the purposes of this invention, “cryptic phase” embraces any non-replicating, intracellular form, of which there are a number of distinct stages, including but not limited to intracellular EBs, EBs transforming into RBs and vice versa, miniature RBs, non-replicating RBs and the like.

As used herein, the term “biological material” refers to bodily secretions, bodily fluids and tissue specimens from a subject (e.g., from a mammal, such as from a human or an animal model). Examples of bodily secretions include, but are not limited to cervical secretions, trachial-bronchial secretions and pharyngeal secretions. Suitable bodily fluids include, but are not limited to blood, sweat, tears, cerebral spinal system fluid, serum, sputum, ear wax, urine, synovial fluid and saliva. Animals, cells and tissue specimens such as from a variety of biopsies are also embraced by this term.

### DETAILED DESCRIPTION

Featured herein are agents that are used in combination to eliminate or interfere with distinct phases of the life cycle of Chlamydia spp. These chlamydial phases include the intracellular metabolizing/replicating phase; the intracellular “cryptic” phases; and the extracellular EB phase. Current concepts of antimicrobial therapy for chlamydial infections address only one phase, the replicating phase. Unless multiple phases of the chlamydial life cycle are addressed by anti-chlamydial therapy, the pathogen is likely to escape the desired effects of the antimicrobial agent(s) and cause recurrent infection after reactivation from latency. The methods described herein are useful for reducing chlamydial infection by reducing or eliminating different phases of chlamydial life cycle, thereby treating inflammatory diseases associated with or resulting from chlamydial infection.

### Agents Effective Against Different Phases of Chlamydial Life Cycle

The methods of treatment and compositions described herein feature a combination of: (a) at least one (e.g., 1, 2, 3, 4, 5, 6, 7, 8, 9, 10, 11, 12, 13, 14, 15, 16, 17, 18, 19, 20, or more) agent effective against (e.g., that can effectively reduce or eliminate) the EB phase of chlamydial life cycle, and (b) at least one (e.g., 1, 2, 3, 4, 5, 6, 7, 8, 9, 10, 11, 12, 13, 14, 15, 16, 17, 18, 19, 20, or more) agent effective against (e.g., that can effectively reduce or eliminate) the cryptic phase and/or replicating phase of chlamydial life cycle. In particular, described herein are methods of reducing or eliminating chlamydial infection, such as chlamydial infection in a subject (e.g., a human), by using a combination of: (a) at least one (e.g., 1, 2, 3, 4, 5, 6, 7, 8, 9, 10, 11, 12, 13, 14, 15, 16, 17, 18, 19, 20, or more) agent effective against (e.g., that can effectively reduce or eliminate) the EB phase of chlamydial life cycle, and (b) at least one (e.g., 1, 2, 3, 4, 5, 6, 7, 8, 9, 10, 11, 12, 13, 14, 15, 16, 17, 18, 19, 20, or more) agent effective against (e.g., that can effectively reduce or eliminate) the cryptic phase and/or replicating phase of chlamydial life cycle. Also described herein are methods of treating an inflammatory disease, such as an inflammatory disease associated with or resulting from chlamydial infection, by using a combination of: (a) at least one (e.g., 1, 2, 3, 4, 5, 6, 7, 8, 9, 10, 11, 12, 13, 14, 15, 16, 17, 18, 19, 20, or more) agent effective against (e.g., that can effectively reduce or eliminate) the EB phase of chlamydial life cycle, and (b) at least one (e.g., 1, 2, 3, 4, 5, 6, 7, 8, 9, 10, 11, 12, 13, 14, 15, 16, 17, 18, 19, 20, or more) agent effective against (e.g., that can effectively reduce or eliminate) the cryptic phase and/or replicating phase of chlamydial life cycle. The therapeutic methods and combination of agents described below are generally applicable for infection caused by any Chlamydia spp, including but not limited to C. pneumoniae, C. trachomatis, C. psittaci, and C. pecorum.

### Agents Effective Against the EB Phase of Chlamydial Life Cycle

The methods of treatment and compositions described herein feature a combination of: (a) at least one (e.g., 1, 2, 3, 4, 5, 6, 7, 8, 9, 10, 11, 12, 13, 14, 15, 16, 17, 18, 19, 20, or more) agent effective against the EB phase of chlamydial life cycle, such as agent that can effectively reduce (e.g., reduce by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more))) or eliminate the EB phase of chlamydial life cycle; and (b) at least one agent effective against the cryptic phase and/or replicating phase of chlamydial life cycle, such as agent that can effectively reduce or eliminate the cryptic phase and/or replicating phase of chlamydial life cycle. In some embodiments, an agent effective against (e.g., that effectively reduces or eliminates) the EB phase of chlamydial life cycle is taurine, or a pharmaceutically acceptable salt or derivative thereof. In particular embodiments, an agent that can effectively reduce or eliminate the EB phase of chlamydial life cycle is N-chlorotaurine (NCT) (e.g., the sodium salt of NCT (Cl—HN—CH₂—CH₂—SO₃—Na)), an N-chloro derivative of the amino acid taurine, such as a 1% (w/v) aqueous solution of NCT. For example, methods and compositions described herein can feature a combination of: (a) taurine (e.g., NCT), such as a sodium salt of NCT) as at least one agent that effectively reduces (e.g., reduce by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more))) or eliminates the EB phase of chlamydial life cycle; and (b) at least one agent effective against (e.g., that effectively reduces or eliminates) the cryptic phase and/or replicating phase of chlamydial life cycle.

Additionally, or alternatively, an agent effective against (e.g., that effectively reduces or eliminates) the EB phase of chlamydial life cycle may be dimethylglycine ((CH₃)₂NCH₂COOH; DMG), a derivative of the amino acid glycine, or a pharmaceutically acceptable salt or derivative thereof. For example, methods and compositions described herein can feature a combination of: (a) dimethylglycine, or a pharmaceutically acceptable salt or derivative thereof, as at least one agent that effectively reduces (e.g., reduce by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more))) or eliminates the EB phase of chlamydial life cycle; and (b) at least one agent effective against (e.g., that effectively reduces or eliminates) the cryptic phase and/or replicating phase of chlamydial life cycle.

Additionally, or alternatively, an agent effective against (e.g., that effectively reduces or eliminates) the EB phase of chlamydial life cycle may be trimethylglycine (TMG; also known as glycine betaine), an amino acid derivative occurring in plants, or a pharmaceutically acceptable salt or derivative thereof. For example, methods and compositions described herein can feature a combination of: (a) trimethylglycine, or a pharmaceutically acceptable salt or derivative thereof, as at least one agent that effectively reduces (e.g., reduce by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more))) or eliminates the EB phase of chlamydial life cycle; and (b) at least one agent effective against (e.g., that effectively reduces or eliminates) the cryptic phase and/or replicating phase of chlamydial life cycle.

In some embodiments, methods of treatment and compositions described herein feature a combination of: (a) taurine or NCT, such as a 1% (w/v) aqueous solution of NCT, or a pharmaceutically acceptable salt or derivative thereof, as an agent effective against (e.g., that effectively reduces or eliminates) the EB phase of chlamydial life cycle; and (b) at least one agent effective against (e.g., that effectively reduces or eliminates) the cryptic phase and/or replicating phase of chlamydial life cycle. In other embodiments, methods of treatment and compositions described herein may feature a combination of: (a) dimethylglycine, or a pharmaceutically acceptable salt or derivative thereof, as an agent effective against (e.g., that effectively reduces or eliminates) the EB phase of chlamydial life cycle; and (b) at least one agent effective against (e.g., that effectively reduces or eliminates) the cryptic phase and/or replicating phase of chlamydial life cycle. Alternatively, methods of treatment and compositions described herein may feature a combination of: (a) trimethylglycine, or a pharmaceutically acceptable salt or derivative thereof, as an agent effective against (e.g., that effectively reduces or eliminates) the EB phase of chlamydial life cycle; and (b) at least one agent effective against (e.g., that effectively reduces or eliminates) the cryptic phase and/or replicating phase of chlamydial life cycle.

In some embodiments, methods of treatment and compositions described herein may feature a combination of: (a) taurine or NCT, such as a 1% (w/v) aqueous solution of NCT, and dimethylglycine, or pharmaceutically acceptable salts or derivatives thereof, as agents effective against (e.g., that effectively reduces or eliminates) the EB phase of chlamydial life cycle; and (b) at least one agent effective against (e.g., that effectively reduces or eliminates) the cryptic phase and/or replicating phase of chlamydial life cycle. In other embodiments, methods of treatment and compositions described herein may feature a combination of: (a) taurine or NCT, such as a 1% (w/v) aqueous solution of NCT, and trimethylglycine, or pharmaceutically acceptable salts or derivatives thereof, as agents effective against (e.g., that effectively reduce or eliminate) the EB phase of chlamydial life cycle; and (b) at least one agent effective against (e.g., that effectively reduces or eliminates) the cryptic phase and/or replicating phase of chlamydial life cycle. In alternative embodiments, methods of treatment and compositions described herein may feature a combination of: (a) dimethylglycine and trimethylglycine, or pharmaceutically acceptable salts or derivatives thereof, as agents effective against (e.g., that effectively reduce or eliminate) the EB phase of chlamydial life cycle; and (b) at least one agent effective against (e.g., that effectively reduces or eliminates) the cryptic phase and/or replicating phase of chlamydial life cycle. Alternatively, methods of treatment and compositions described herein may feature a combination of: (a) taurine or NCT, such as a 1% (w/v) aqueous solution of NCT, dimethylglycine and trimethylglycine, or pharmaceutically acceptable salts or derivatives thereof, as agents effective against (e.g., that effectively reduce or eliminate) the EB phase of chlamydial life cycle; and (b) at least one agent effective against (e.g., that effectively reduces or eliminates) the cryptic phase and/or replicating phase of chlamydial life cycle.

Agents Effective Against the Cryptic Phase and/or Replicating Phase of Chlamydial Life Cycle

The methods of treatment and compositions described herein feature a combination of: (a) at least one agent effective against (e.g., that can effectively reduce or eliminate) the EB phase of chlamydial life cycle; and (b) at least one (e.g., 1, 2, 3, 4, 5, 6, 7, 8, 9, 10, 11, 12, 13, 14, 15, 16, 17, 18, 19, 20, or more) agent effective against the cryptic phase and/or replicating phase of chlamydial life cycle, such as agent that can effectively reduce (e.g., reduce by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more))) or eliminate the cryptic phase and/or replicating phase of chlamydial life cycle. In some embodiments, an agent effective against (e.g., that effectively reduces or eliminates) the cryptic phase and/or replicating phase of chlamydial life cycle is a rifamycin antibiotic, such as rifabutin (MYCOBUTIN®), rifampicin (RIFADIN®), or rifapentin (PRIFTIN®). In particular embodiments, an agent that can effectively reduce or eliminate the cryptic phase and/or replicating phase of chlamydial life cycle is rifabutin (MYCOBUTIN®). For example, methods and compositions described herein can feature a combination of: (a) at least one agent effective against (e.g., that effectively reduces or eliminates) the EB phase of chlamydial life cycle; and (b) a rifamycin antibiotic (e.g., rifabutin, rifampicin, or rifapentin) as at least one agent that effectively reduces (e.g., reduces by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more))) or eliminates the cryptic phase and/or replicating phase of chlamydial life cycle.

Additionally or alternatively, an agent that effective against (e.g., effectively reduces or eliminates) the cryptic phase and/or replicating phase of chlamydial life cycle is a macrolide antibiotic, such as azithromycin (e.g., ZITHROMAX®, TRI-PAK®, ZMAX®, ZITHROMAX®, ZITHROMAX Z-PAK®, AZASITE®, etc.), erythromycin (e.g., E.E.S. 400®, E.E.S. GRANULES®, ERYPED 200®, ERYPED 400®, ERY PADS®, ERYGEL®, ERYC®, PCE®, ERY-TAB®, etc.), clarithromycin (e.g., BIAXIN®, BIAXIN XL®, etc.), spiramycin (ROVAMYCINE®), oleandomycin (MASTALONE®, MASTIGUARD®), josamycin (JOSALID®, JOSACINE®, IOSALIDE®, JOSAMINA®, WILPRAFEN®, JOSAMY®), kitsamysin (e.g., KITASAMYCIN 11%®, KITKAN®, LIKEJHN®, LE PU MEI XIN®, LEUCOMYCIN®, SHUANG NING®, WAN JI®, XIAOJUN®, CEVA KITASAMYCIN®, KITASAMYCIN-RUI YANG PHARM®, LEUCOMYCIN®, LEUKOMYCIN®, etc.), flurithromycin (e.g., FLURIZIC®, MIZAR®, etc.) or roxithromycin (e.g., ACEVOR®, AMMIROX®, ARISTOMYCIN®, ASSORAL®, AZURIL®, BAZUCTRIL®, BIAXSIG®, BICOFEN®, CIRUMYCIN (FM)®, CLARAMID®, CLARAMID®, CROLIX®, DELITROXIN®, EROXADE®, ERYBROS®, FLOXID®, FORILIN (FM)®, FORIMYCIN®, INFECTOROXIT®, KENSODIC®, MACROSIL®, NEO-SUXIGAL®, OVERAL®, POLIROXIN®, RAMIVAN®, REDOTRIN®, RENICIN®, ROMICIN®, ROSSITROL®, ROTESAN (FM)®, ROTHRICIN®, ROTRAM®, ROTRAMIN®, ROVENAL®, ROXCIN®, ROXI®, ROXIBETA®, ROXIBION®, ROXICILLINE®, ROXICIN®, ROXID®, ROXIDURA®, ROXIGAMMA®, ROXIGRUN®, ROXIHEFA®, ROXIHEXAL®, ROXIKLINGE (FM)®, ROXILAN®, ROXIMIN®, ROXIMIN-GALENICA®, ROXIMSTAD®, ROXINA®, ROXINOX®, ROXI-PAED®, ROXI-PUREN®, ROXI-Q®, ROXI-SAAR®, ROXITHRO-LICH®, ROXITHROSTAD®, ROXITHROXYL®, ROXITIN®, ROXITRAN®, ROXITRICINA®, ROXITROL (FM)®, ROXITROM®, ROXITROMIN®, ROXLECON®, ROXO®, ROXOMYCIN®, ROXTHOMED®, ROXTHRIN®, ROXTO®, ROXTROCIN®, ROXY (DI)®, RUCIN®, RULID®, RULIDE®, SEIDE®, SUBROXINE®, SURLID®, TIRABICIN®, TOSCAMYCIN-R®, UONIN®, URAMILON®, UTOLID®, VASELPIN®, VOMITORAN®, etc.). In particular embodiments, an agent that can effectively reduce or eliminate the cryptic phase and/or replicating phase of chlamydial life cycle is azithromycin (e.g., ZITHROMAX®, TRI-PAK®, ZMAX®, ZITHROMAX®, ZITHROMAX Z-PAK®, AZASITE®, etc.). For example, methods and compositions described herein can feature a combination of: (a) at least one agent effective against (e.g., that effectively reduces or eliminates) the EB phase of chlamydial life cycle; and (b) a macrolide antibiotic (e.g., azithromycin, erythromycin, clarithromycin, roxithromycin, spiramycin, oleandomycin, josamycin, kitsamysin, or flurithromycin) as at least one agent that effectively reduces (e.g., reduces by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more))) or eliminates the cryptic phase and/or replicating phase of chlamydial life cycle.

Additionally or alternatively, an agent effective against (e.g., that effectively reduces or eliminates) the cryptic phase and/or replicating phase of chlamydial life cycle is a tetracycline antibiotic, such as minocycline (e.g., MINOCIN®, DYNACIN®, SOLODYN®, etc.), doxycycline (e.g., ORACEA®, TARGADOX®, DORYX MPC®, MORGIDOX®, DORYX®, MONODOX®, AVIDOXY®, MORGIDOX 1X100®, ACTICLATE®, MONDOXYNE NL®, etc.), chlortetracycline (AUREOMYCIN®), tetracycline hydrochloride (SUMYCIN®), oxytetracycline (TERRAMYCIN®), demeclocycline (DECLOMYCIN®), or methacycline (e.g., BIALATAN®, METACICLINA®, etc.). In particular embodiments, an agent that can effectively reduce or eliminate the cryptic phase and/or replicating phase of chlamydial life cycle is minocycline (e.g., MINOCIN®, DYNACIN®, SOLODYN®, etc.). For example, methods and compositions described herein can feature a combination of: (a) at least one agent effective against (e.g., that effectively reduces or eliminates) the EB phase of chlamydial life cycle; and (b) a tetracycline antibiotic (e.g., minocycline, doxycycline, chlortetracycline, tetracycline hydrochloride, oxytetracycline, demeclocycline, or methacycline) as at least one agent that effectively reduces (e.g., reduces by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more))) or eliminates the cryptic phase and/or replicating phase of chlamydial life cycle.

In some embodiments, methods of treatment and compositions described herein feature a combination of: (a) at least one agent effective against (e.g., that effectively reduces or eliminates) the EB phase of chlamydial life cycle (e.g., taurine; dimethylglycine; trimethylglycine; taurine and dimethylglycine; taurine and trimethylglycine; dimethylglycine and trimethylglycine; or taurine, dimethylglycine and trimethylglycine); and (b) a rifamycin antibiotic (e.g., rifabutin, rifampicin, or rifapentin) as an agent effective against (e.g., that effectively reduces or eliminates) the cryptic phase and/or replicating phase of chlamydial life cycle. In other embodiments, methods of treatment and compositions described herein feature a combination of: (a) at least one agent effective against (e.g., that effectively reduces or eliminates) the EB phase of chlamydial life cycle (e.g., taurine; dimethylglycine; trimethylglycine; taurine and dimethylglycine; taurine and trimethylglycine; dimethylglycine and trimethylglycine; or taurine, dimethylglycine and trimethylglycine); and (b) a macrolide antibiotic (e.g., azithromycin, erythromycin, clarithromycin, roxithromycin, spiramycin, oleandomycin, josamycin, kitsamysin, or flurithromycin) as an agent effective against (e.g., that effectively reduces or eliminates) the cryptic phase and/or replicating phase of chlamydial life cycle. Alternatively, methods of treatment and compositions described herein may feature a combination of: (a) at least one agent effective against (e.g., that effectively reduces or eliminates) the EB phase of chlamydial life cycle (e.g., taurine; dimethylglycine; trimethylglycine; taurine and dimethylglycine; taurine and trimethylglycine; dimethylglycine and trimethylglycine; or taurine, dimethylglycine and trimethylglycine); and (b) a tetracycline antibiotic (e.g., minocycline, doxycycline, chlortetracycline, tetracycline hydrochloride, oxytetracycline, demeclocycline, or methacycline) as an agent effective against (e.g., that effectively reduces or eliminates) the cryptic phase and/or replicating phase of chlamydial life cycle.

In some embodiments, methods of treatment and compositions described herein feature a combination of: (a) at least one agent effective against (e.g., that effectively reduces or eliminates) the EB phase of chlamydial life cycle (e.g., taurine; dimethylglycine; trimethylglycine; taurine and dimethylglycine; taurine and trimethylglycine; dimethylglycine and trimethylglycine; or taurine, dimethylglycine and trimethylglycine); and (b) a rifamycin antibiotic (e.g., rifabutin, rifampicin, or rifapentin) and a macrolide antibiotic (e.g., azithromycin, erythromycin, clarithromycin, roxithromycin, spiramycin, oleandomycin, josamycin, kitsamysin, or flurithromycin) as agents effective against (e.g., that effectively reduce or eliminate) the cryptic phase and/or replicating phase of chlamydial life cycle. In other embodiments, methods of treatment and compositions described herein feature a combination of: (a) at least one agent effective against (e.g., that effectively reduces or eliminates) the EB phase of chlamydial life cycle (e.g., taurine; dimethylglycine; trimethylglycine; taurine and dimethylglycine; taurine and trimethylglycine; dimethylglycine and trimethylglycine; or taurine, dimethylglycine and trimethylglycine); and (b) a rifamycin antibiotic (e.g., rifabutin, rifampicin, or rifapentin) and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle. In alternative embodiments, methods of treatment and compositions described herein may feature a combination of: (a) at least one agent effective against (e.g., that effectively reduces or eliminates) the EB phase of chlamydial life cycle (e.g., taurine; dimethylglycine; trimethylglycine; taurine and dimethylglycine; taurine and trimethylglycine; dimethylglycine and trimethylglycine; or taurine, dimethylglycine and trimethylglycine); and (b) a macrolide antibiotic (e.g., azithromycin, erythromycin, clarithromycin, roxithromycin, spiramycin, oleandomycin, josamycin, kitsamysin, or flurithromycin) and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle. Alternatively, methods of treatment and compositions described herein may feature a combination of: (a) at least one agent effective against (e.g., that effectively reduces or eliminates) the EB phase of chlamydial life cycle (e.g., taurine; dimethylglycine; trimethylglycine; taurine and dimethylglycine; taurine and trimethylglycine; dimethylglycine and trimethylglycine; or taurine, dimethylglycine and trimethylglycine); and (b) a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle.

In particular, methods of treatment and compositions described herein may feature a combination of: (a) taurine or NCT, such as a 1% (w/v) aqueous solution of NCT, as an agent effective against (e.g., that effectively reduces or eliminates) the EB phase of chlamydial life cycle; and (b) a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle. Alternatively, methods of treatment and compositions described herein may feature a combination of: (a) dimethylglycine as an agent effective against (e.g., that effectively reduces or eliminates) the EB phase of chlamydial life cycle; and (b) a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle. Alternatively, methods of treatment and compositions described herein may feature a combination of: (a) trimethylglycine as an agent effective against (e.g., that effectively reduces or eliminates) the EB phase of chlamydial life cycle; and (b) a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle. Alternatively, methods of treatment and compositions described herein may feature a combination of: (a) taurine or NCT, such as a 1% (w/v) aqueous solution of NCT, and dimethylglycine as agents effective against (e.g., that effectively reduce or eliminate) the EB phase of chlamydial life cycle; and (b) a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle. Alternatively, methods of treatment and compositions described herein may feature a combination of: (a) taurine or NCT, such as a 1% (w/v) aqueous solution of NCT, and trimethylglycine as agents effective against (e.g., that effectively reduce or eliminate) the EB phase of chlamydial life cycle; and (b) a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle. Alternatively, methods of treatment and compositions described herein may feature a combination of: (a) dimethylglycine and trimethylglycine as agents effective against (e.g., that effectively reduce or eliminate) the EB phase of chlamydial life cycle; and (b) a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle. Alternatively, methods of treatment and compositions described herein may feature a combination of: (a) taurine or NCT, such as a 1% (w/v) aqueous solution of NCT, dimethylglycine and trimethylglycine as agents effective against (e.g., that effectively reduce or eliminate) the EB phase of chlamydial life cycle; and (b) a rifamycin antibiotic (e.g., rifabutin, rifampicin, or rifapentin), a macrolide antibiotic (e.g., azithromycin, erythromycin, clarithromycin, roxithromycin, spiramycin, oleandomycin, josamycin, kitsamysin, or flurithromycin), and a tetracycline antibiotic (e.g., minocycline, doxycycline, chlortetracycline, tetracycline hydrochloride, oxytetracycline, demeclocycline, or methacycline) as agents effective against (e.g., that effectively reduce or eliminate) the cryptic phase and/or replicating phase of chlamydial life cycle.

### Reduction or Elimination of Chlamydial Infection

Methods and compositions described herein (e.g., a combination of: (a) at least one agent effective against (e.g., that effectively reduces or eliminates) the EB phase of chlamydial life cycle (e.g., one or more of taurine or an analog or derivative thereof (e.g., NCT), such as a 1% (w/v) aqueous solution of NCT, dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic); and method of treatment therewith) can be used for reduction or elimination of chlamydial infection (e.g., infection caused by any Chlamydia spp., including but not limited to C. pneumoniae, C. trachomatis, C. psittaci, and C. pecorum), such as chlamydial infection in a subject (e.g., a mammal, such as a human (e.g., a patient with chlamydial infection) or an animal model) or a cell culture (e.g., a culture of mammalian cells). The compositions and methods described herein can eliminate or reduce chlamydial infection in a subject (e.g., a mammal, such as a human or an animal model) or a cell culture (e.g., a culture of mammalian cells) by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more)) compared to chlamydial infection in the subject or cell culture before administration of the combination of agents described herein or compared to chlamydial infection in a reference subject or cell culture (e.g., a subject or cell culture that is not treated with the combination of agents described herein). The compositions and methods described herein can eliminate or reduce chlamydial infection by eliminating or reducing the different phases of chlamydial life cycle (e.g., the EB phase of chlamydial life cycle, the cryptic phase of chlamydial life cycle, the replicating phase of chlamydial life cycle) in a subject (e.g., in a biological material (e.g., bodily secretion, bodily fluid, and/or tissue) from a subject, such as in a biological material from a mammal) or a cell culture (e.g., a culture of mammalian cells) by 5% or more (e.g., between 5-20%, between 5-50%, between 10-50%, between 10-80%, between 20-80%, or between 20-100% (e.g., by 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more)) compared to the different phases of chlamydial life cycle in the subject or cell culture before administration of the combination of agents described herein or compared to the different phases of chlamydial life cycle in a reference subject or cell culture (e.g., a subject or cell culture that is not treated with the combination of agents described herein).

Reduction or elimination of chlamydial infection and/or reduction or elimination of the different phases of chlamydial life cycle (e.g., the EB phase of chlamydial life cycle, the cryptic phase of chlamydial life cycle, and the replicating phase of chlamydial life cycle) in a subject (e.g., in a biological material from a subject, such as in a biological material (e.g., bodily secretion, bodily fluid, and/or tissue) from a mammal) or a cell culture (e.g., a culture of mammalian cells) can be determined by one or more diagnostic methods, such as tests (e.g., assays) that detect chlamydial nucleic acids or proteins. One or more of such diagnostic methods are described in US 2003/0171348, which is incorporated herein by reference in its entirety.

In particular, reduction or elimination of chlamydial infection in a subject (e.g., a mammal) or a cell culture (e.g., a culture of mammalian cells) can be determined by one or more diagnostic methods, such as tests (e.g., assays) that detect chlamydial nucleic acids or proteins in a biological material (e.g., bodily secretion, bodily fluid, and/or tissue) from the subject or the cell culture. Also, reduction or elimination of the different phases of chlamydial life cycle (e.g., the EB phase of chlamydial life cycle, the cryptic phase of chlamydial life cycle, and the replicating phase of chlamydial life cycle) in a subject (e.g., a mammal) or a cell culture (e.g., a culture of mammalian cells) can be determined by one or more diagnostic methods, such as tests (e.g., assays) that detect chlamydial nucleic acids or proteins in a biological material (e.g., bodily secretion, bodily fluid, and/or tissue) from the subject or the cell culture. In some embodiments, such diagnostic methods (e.g., tests, such as assays) may include polymerase chain reactions, enzyme-linked immunosorbent assays (ELISA), luminescence assays (e.g., fluorescence and chemiluminescence), radioimmunoassay, and immunohistology. In particular, tests (e.g., assays) that detect chlamydial nucleic acids or proteins encoded thereby in a biological material from a subject or a cell culture may include polymerase chain reactions, and immunological methods such as enzyme-linked immunosorbent assays (ELISA), including luminescence assays (e.g., fluorescence and chemiluminescence), radioimmunoassay, and immunohistology. For example, chlamydial infection and/or different phases of chlamydial life cycle (e.g., the EB phase of chlamydial life cycle, the cryptic phase of chlamydial life cycle, and the replicating phase of chlamydial life cycle) in a subject (e.g., a mammal) or a cell culture (e.g., a culture of mammalian cells) can be determined by detecting chlamydial nucleic acids or proteins in a biological material (e.g., bodily secretion, bodily fluid, and/or tissue) from the subject or the cell culture by one or more tests or assays (e.g., polymerase chain reactions, ELISA, luminescence assays (e.g., fluorescence and chemiluminescence), radioimmunoassay, and/or immunohistology). A subject (e.g., a biological material from a subject) or a cell culture is considered to test negative for chlamydial infection when chlamydial nucleic acid or protein in the subject (e.g., in the biological material from the subject) or in the cell culture is not detected or is undetectable by a diagnostic method described herein (e.g., test or assay to detect chlamydial nucleic acid or protein); or when the amount of chlamydial nucleic acid or protein in the subject (e.g., in the biological material from the subject) or in the cell culture falls below the diagnostic method's lower threshold of detection.

In some embodiments, reduction or elimination of chlamydial infection and/or the different phases of chlamydial life cycle can be determined by comparing the amount of chlamydial nucleic acids or proteins in a subject (e.g., in a biological material (e.g., bodily secretion, bodily fluid, and/or tissue) from a subject) or a cell culture following treatment with the methods and compositions described herein (e.g., following administration of a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) to the amount of chlamydial nucleic acids or proteins in the subject or cell culture before administration of the combination of agents described herein, or to the amount of chlamydial nucleic acids or proteins in a reference subject or cell culture (e.g., a subject or cell culture that is not treated with the combination of agents described herein). The methods and compositions described herein are considered to eliminate chlamydial infection and/or the different phases of chlamydial life cycle, when, following treatment with the methods and compositions described herein (e.g., following administration of a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)), the amount of chlamydial nucleic acids or proteins in a subject (e.g., in a biological material (e.g., bodily secretion, bodily fluid, and/or tissue) from a subject) or a cell culture reaches an undetectable level (e.g., not detected by the diagnostic methods, such as tests described herein); such as, when following treatment with the methods and compositions described herein, the subject (e.g., a biological material from the subject) or cell culture tests negative for chlamydial infection by the diagnostic methods described herein.

### Treatment of Inflammatory Diseases

Methods and compositions described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic); and method of treatment therewith) may be used for treating chronic inflammatory diseases, such as inflammatory diseases associated with or resulting from chlamydial infection (e.g., infection by any Chlamydia spp., including but not limited to C. pneumoniae, C. trachomatis, C. psittaci, and C. pecorum), in a subject (e.g., a mammal, such as a human (e.g., a patient with chlamydial infection) or an animal model) in need thereof.

In some embodiments, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)), inhibits or reduces inflammation (e.g., inhibits or reduces release of inflammatory cytokines (e.g., TNF-α, IFNγ, IL-1, IL-6, IL-1β, IL-12, IL-18, etc.) from cells) in a subject (e.g., a human subject or an animal model) or in a cell culture (e.g., a culture of mammalian cells), when the combination of agents described herein is administered to the subject or to the cell culture in an amount (e.g., an effective amount) and for a time sufficient to inhibit or reduce inflammation (e.g., inhibit or reduce release of inflammatory cytokines). Administration of a combination of agents described herein can inhibit or reduce inflammation by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before administration of the combination of agents or compared to a reference subject or cell culture to which the combination of agents is not administered.

In some embodiments, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)), reduces one or more symptoms of an inflammatory disease in a subject (e.g., a human subject or an animal model with the disease), when the combination of agents described herein is administered to the subject in an amount (e.g., an effective amount) and for a time sufficient to reduce the symptoms of the inflammatory disease. Administration of a combination of agents described herein can reduce symptoms of an inflammatory disease by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before administration of the combination of agents or compared to a reference subject to which the combination of agents is not administered.

Inflammatory disease that can be treated by the methods and compositions described herein can be a respiratory disease, a neurodegenerative disease, a musculoskeletal disease, or a cardiovascular disease.

### Treatment of Respiratory Diseases

In some embodiments, the methods and compositions described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic); and method of treatment therewith) can be used for treating a respiratory disease, such as a respiratory disease associated with or resulting from chlamydial infection (e.g., infection by any Chlamydia spp., including but not limited to C. pneumoniae, C. trachomatis, C. psittaci, and C. pecorum), in a subject (e.g., a mammal, such as a human) in need thereof. Respiratory diseases that can be treated by the methods and compositions described herein include, but are not limited to: respiratory condition involving inflammation or infection of a respiratory mucosa; respiratory condition involving inflammation or infection of an underlying muscle of the respiratory tract; cystic fibrosis; pneumonia; asthma; bronchitis; sinusitis or rhinosinusitis; infection of a sinus; chronic obstructive airway disease; emphysema; chronic bronchitis; and bronchiectasis. In particular, a respiratory disease that can be treated by the methods and compositions described herein may be cystic fibrosis, pneumonia, or asthma. For example, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) can be used for treating respiratory condition involving inflammation or infection of a respiratory mucosa, respiratory condition involving inflammation or infection of an underlying muscle of the respiratory tract, cystic fibrosis, pneumonia, asthma, bronchitis, sinusitis or rhinosinusitis, infection of a sinus, chronic obstructive airway disease, emphysema, chronic bronchitis, or bronchiectasis in a subject in need thereof.

In some embodiments, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)), reduces one or more symptoms of a respiratory disease (e.g., respiratory condition involving inflammation or infection of a respiratory mucosa, respiratory condition involving inflammation or infection of an underlying muscle of the respiratory tract, cystic fibrosis, pneumonia, asthma, bronchitis, sinusitis or rhinosinusitis, infection of a sinus, chronic obstructive airway disease, emphysema, chronic bronchitis, or bronchiectasis) in a subject (e.g., a human subject or an animal model with the disease), when the combination of agents described herein is administered to the subject in an amount (e.g., an effective amount) and for a time sufficient to reduce the symptoms of the respiratory disease. Administration of a combination of agents described herein can reduce symptoms of a respiratory disease by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before administration of the combination of agents or compared to a reference subject to which the combination of agents is not administered.

### Treatment of Neurodegenerative Diseases

In some embodiments, the methods and compositions described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic); and method of treatment therewith) can be used for treating a neurodegenerative disease, such as a neurodegenerative disease associated with or resulting from chlamydial infection (e.g., infection by any Chlamydia spp., including but not limited to C. pneumoniae, C. trachomatis, C. psittaci, and C. pecorum), in a subject (e.g., a mammal, such as a human) in need thereof. Neurodegenerative diseases that can be treated by the methods and compositions described herein include, but are not limited to Alzheimer's disease, Parkinson's disease, Prion disease, motor neuron diseases, Huntington's disease, spinocerebellar ataxia, and spinal muscular atrophy. In particular, a neurodegenerative disease that can be treated by the methods and compositions described herein may be Alzheimer's disease, Multiple Sclerosis, and dementia. For example, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) can be used for treating Alzheimer's disease, Parkinson's disease, Prion disease, motor neuron diseases, Huntington's disease, spinocerebellar ataxia, or spinal muscular atrophy in a subject in need thereof.

In some embodiments, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)), reduces one or more symptoms of a neurodegenerative disease (e.g., Alzheimer's disease, Parkinson's disease, Prion disease, motor neuron diseases, Huntington's disease, spinocerebellar ataxia, or spinal muscular atrophy) in a subject (e.g., a human subject or an animal model with the disease), when the combination of agents described herein is administered to the subject in an amount (e.g., an effective amount) and for a time sufficient to reduce the symptoms of the neurodegenerative disease. Administration of a combination of agents described herein can reduce symptoms of a neurodegenerative disease by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before administration of the combination of agents or compared to a reference subject to which the combination of agents is not administered.

### Treatment of Musculoskeletal Diseases

In some embodiments, the methods and compositions described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic); and method of treatment therewith) can be used for treating a musculoskeletal disease, such as a musculoskeletal disease associated with or resulting from chlamydial infection (e.g., infection by any Chlamydia spp., including but not limited to C. pneumoniae, C. trachomatis, C. psittaci, and C. pecorum), in a subject (e.g., a mammal, such as a human) in need thereof. Musculoskeletal diseases that can be treated by the methods and compositions described herein include, but are not limited to arthritis (e.g., reactive arthritis, osteoarthritis, or rheumatoid arthritis), tendinitis, carpal tunnel syndrome, and fibromyalgia. In particular, a musculoskeletal disease that can be treated by the methods and compositions described herein may be arthritis (e.g., reactive arthritis, osteoarthritis, or rheumatoid arthritis). For example, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) can be used for treating arthritis (e.g., reactive arthritis, osteoarthritis, or rheumatoid arthritis), tendinitis, carpal tunnel syndrome, or fibromyalgia in a subject in need thereof.

In some embodiments, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)), reduces one or more symptoms of a musculoskeletal disease (e.g., arthritis (e.g., reactive arthritis, osteoarthritis, or rheumatoid arthritis), tendinitis, carpal tunnel syndrome, or fibromyalgia) in a subject (e.g., a human subject or an animal model with the disease), when the combination of agents described herein is administered to the subject in an amount (e.g., an effective amount) and for a time sufficient to reduce the symptoms of the musculoskeletal disease. Administration of a combination of agents described herein can reduce symptoms of a musculoskeletal disease by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before administration of the combination of agents or compared to a reference subject to which the combination of agents is not administered.

### Treatment of Cardiovascular Diseases

In some embodiments, the methods and compositions described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic); and method of treatment therewith) can be used for treating a cardiovascular disease, such as a cardiovascular disease associated with or resulting from chlamydial infection (e.g., infection by any Chlamydia spp., including but not limited to C. pneumoniae, C. trachomatis, C. psittaci, and C. pecorum), in a subject (e.g., a mammal, such as a human) in need thereof. Cardiovascular diseases that can be treated by the methods and compositions described herein include, but are not limited to atherosclerosis, coronary artery diseases, stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis. In particular, a cardiovascular disease that can be treated by the methods and compositions described herein may be atherosclerosis. For example, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) can be used for treating atherosclerosis, coronary artery diseases, stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, or venous thrombosis in a subject in need thereof.

In some embodiments, the methods and compositions described herein (e.g., treatment with a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)), reduces one or more symptoms of a cardiovascular disease (e.g., atherosclerosis, coronary artery diseases, stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, or venous thrombosis) in a subject (e.g., a human subject or an animal model with the disease), when the combination of agents described herein is administered to the subject in an amount (e.g., an effective amount) and for a time sufficient to reduce the symptoms of the cardiovascular disease. Administration of a combination of agents described herein can reduce symptoms of a cardiovascular disease by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before administration of the combination of agents or compared to a reference subject to which the combination of agents is not administered.

### Formulations and Carriers

In order to be administered to a subject (e.g., a human subject or an animal model), agents (e.g., one or more of taurine or an analog or derivative thereof (e.g., NCT), dimethylglycine, trimethylglycine, rifamycin antibiotic, macrolide antibiotic, and tetracycline antibiotic) or combinations thereof described herein, can be formulated as pharmaceutical compositions. In some embodiments, the agents described herein (e.g., taurine, dimethylglycine, trimethylglycine, a rifamycin antibiotic, a macrolide antibiotic, or a tetracycline antibiotic) can be formulated individually as separate pharmaceutical compositions or formulations. In other embodiments, a combination of agents (e.g., a combination of: (a) one or more of taurine or an analog or derivative thereof (e.g., NCT), dimethylglycine, and trimethylglycine; and (b) one or more of a rifamycin antibiotic, a macrolide antibiotic, and a tetracycline antibiotic) can be formulated as a single pharmaceutical composition or formulation. In some embodiments, a pharmaceutical composition or formulation contemplated herein includes a single agent (e.g., taurine, NCT, dimethylglycine, trimethylglycine, a rifamycin antibiotic, a macrolide antibiotic, or a tetracycline antibiotic). In other embodiments, a pharmaceutical composition or formulation contemplated herein includes more than one agent (e.g., two, three, four, five, or more agents), such as a combination of two or more of taurine, dimethylglycine, trimethylglycine, a rifamycin antibiotic, a macrolide antibiotic, and a tetracycline antibiotic. In some embodiments, a pharmaceutical composition or formulation contemplated herein includes a combination of: (a) one or more of taurine or an analog or derivative thereof (e.g., NCT), dimethylglycine, and trimethylglycine; and (b) one or more of a rifamycin antibiotic, a macrolide antibiotic, and a tetracycline antibiotic.

Any pharmaceutical composition or formulation contemplated herein may further include a pharmaceutically acceptable carrier, adjuvant or vehicle. A pharmaceutically acceptable carrier or excipient refers to a carrier (e.g., carrier, media, diluent, solvent, vehicle, etc.) which does not significantly interfere with the biological activity or effectiveness of the active ingredient(s) of a pharmaceutical composition (e.g., active ingredient(s) of one or more of taurine or an analog or derivative thereof (e.g., NCT), dimethylglycine, trimethylglycine, rifamycin antibiotic, macrolide antibiotic, or tetracycline antibiotic) and which is not excessively toxic to the host at the concentrations at which it is used or administered. Pharmaceutical compositions or formulations contemplated herein may include carriers (e.g., diluents, excipients and auxiliaries) that facilitate processing of the active components (e.g., active ingredient(s) of taurine, dimethylglycine, trimethylglycine, rifamycin antibiotic, macrolide antibiotic, or tetracycline antibiotic) into a pharmaceutically acceptable formulation. Carriers employed may be either solid or liquid. Exemplary solid carriers are lactose, sucrose, talc, gelatin, agar, pectin, acacia, magnesium stearate, stearic acid, and the like. Exemplary liquid carriers are syrup, peanut oil, olive oil, water, and the like. Similarly, the pharmaceutical compositions may include time-delay or time-release material known in the art, such as glyceryl monostearate or glyceryl distearate alone or with a wax, ethylcellulose, hydroxypropylmethylcellulose, methylmethacrylate, or the like. Further additives or excipients may be added to achieve the desired formulation properties. For example, a bioavailability enhancer, such: as LABRASOL®, GELUCIRE®, or the like, or formulators, such as CHIC (carboxy-methylcellulose), PG (propyleneglycol), or PEG (polyethyleneglycol), may be added. GELUCIRE®, a semi-solid vehicle that protects active ingredients from light, moisture and oxidation, may be added, e.g., when preparing a capsule formulation. Other pharmaceutically acceptable ingredients can be present in the composition as well. Suitable substances and their use for the formulation of pharmaceutically active compounds are well-known in the art (see, for example, Remington: The Science and Practice of Pharmacy 22^{th} edition (2012), for additional discussion of pharmaceutically acceptable substances and methods of preparing pharmaceutical compositions of various types).

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of ointment, lotion, gel, cream, salve, liniment, paste, tonic, unguent, spray, soap, shampoo, lip balm, syrup, emulsion, soft gelatin capsule, sterile injectable solution or suspension in an ampoule or vial or non-aqueous liquid suspension. Further, the pharmaceutical composition may be incorporated into a skin patch for delivery of the drug directly onto the skin. The inventive compositions are prepared in unit-dosage form appropriate for the mode of administration, e.g., parenteral (e.g., topical) or oral administration.

To obtain a stable water-soluble dose form, the active components of the present invention (e.g., active component of one or more of taurine or an analog or derivative thereof (e.g., NCT), dimethylglycine, trimethylglycine, rifamycin antibiotic, macrolide antibiotic, or tetracycline antibiotic) may be dissolved in water, or an aqueous solution of an organic or inorganic acid, such as 0.3 M solution of succinic acid or citric acid. The active components may also be dissolved in a suitable co-solvent or combinations of co-solvents. Examples of suitable co-solvents include alcohol, propylene glycol, polyethylene glycol 300, polysorbate 80, glycerin, and the like in concentrations ranging from 0-60% of the total volume. For example, an active component of the present invention can be dissolved in DMSO and diluted with water. The composition may also be in the form of a solution of a salt form of the active ingredient in an appropriate aqueous vehicle such as water or isotonic saline or dextrose solution.

Pharmaceutically acceptable carriers, adjuvants and vehicles that may be used in the pharmaceutical compositions or formulations of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethyleneglycol, sodium carboxymethylcellulose, polyacrylates, waxes, polyethylene-polyoxypropylene-block polymers, polyethylene glycol, liposomes and wool fat.

The composition of this invention may be modified by appending appropriate functionalities to enhance selective biological properties. Such modifications are known in the art and include those that increase biological penetration into a given biological system (e.g., blood, lymphatic system, central nervous system), increase oral bioavailability, increase solubility to allow administration by injection, alter metabolism, or alter rate of excretion (Pharmacokinetic Optimization in Drug Research, Testa, B. et al., 2001, Wiley-VCH, VCHA).

A pharmaceutical composition is typically formulated to be compatible with its intended route of administration. The pharmaceutical compositions of this invention may be administered parenterally (e.g., topically), orally, by inhalation spray, rectally, nasally, buccally, vaginally, or via an implanted reservoir, and are preferably administered orally. The pharmaceutical compositions of this invention may contain any conventional non-toxic pharmaceutically acceptable carriers, adjuvants or vehicles. The term “parenteral” or “parenterally” as used herein includes topical administration, injections (e.g., sub-cutaneous, intra-cutaneous, intra-venous, intra-muscular, intra-articular, intra-synovial, intra-sternal, intra-thecal, intra-lesional and intracranial injection) or infusion techniques.

For oral administration, agents (e.g., one or more of taurine or an analog or derivative thereof (e.g., NCT), dimethylglycine, trimethylglycine, rifamycin antibiotic, macrolide antibiotic, or tetracycline antibiotic) can be formulated by combining the active compounds with pharmaceutically acceptable carriers well known in the art. Such carriers enable the agent(s) or combinations thereof to be formulated as a powder, tablet, pill, capsule, lozenge, liquid, gel, syrup, slurry, suspension, and the like. It is recognized that some pharmaceutical compositions, if administered orally, must be protected from digestion. This is typically accomplished either by complexing the agent(s) or combination thereof with a composition to render it resistant to acidic and enzymatic hydrolysis or by packaging the agent in an appropriately resistant carrier such as a liposome. Suitable excipients for oral dosage forms include, for example, fillers such as sugars, including lactose, sucrose, mannitol, or sorbitol; cellulose preparations such as, for example, starch, gelatin, gum tragacanth, methyl cellulose, hydroxypropylmethyl cellulose, sodium carboxymethylcellulose, and/or polyvinylpyrrolidone (PVP). Disintegrating agents may be added, such as the cross linked polyvinyl pyrrolidone, agar, or alginic acid or a salt thereof such as sodium alginate. Lubricating agents, such as magnesium stearate, are also typically added. When aqueous suspensions are administered orally, the active ingredient is combined with emulsifying and suspending agents. If desired, certain sweetening, flavoring, and/or coloring agents may be added. Optionally the oral formulations may also be formulated in saline or buffers for neutralizing internal acid conditions or may be administered without any carriers.

For administration by inhalation, pharmaceutical compositions of this invention may be formulated in the form of an aerosol spray from a pressured container or dispenser, which contains a suitable propellant, e.g., a gas such as carbon dioxide, a fluorocarbon, or a nebulizer. Liquid or dry aerosol (e.g., dry powders, large porous particles, etc.) can also be used. The pharmaceutical compositions of this invention may be administered by nasal aerosol or inhalation. Such compositions are prepared according to techniques well-known in the art of pharmaceutical formulation and may be prepared as solutions in saline employing benzyl alcohol or other suitable preservatives, absorption promoters to enhance bioavailability, fluorocarbons, and/or other solubilizing or dispersing agents known in the art.

For administration by injection, pharmaceutical compositions of this invention can be formulated in the form of a sterile injectable preparation, for example, as a sterile injectable aqueous or oleaginous suspension using a sterile solution or any pharmaceutically acceptable liquid as a vehicle. This suspension may be formulated according to techniques known in the art using suitable dispersing or wetting agents (e.g., Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solutions. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. Pharmaceutically acceptable vehicles also include, but are not limited to, cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

For topical application, a pharmaceutical composition may be formulated in a suitable ointment, lotion, gel, cream, salve, liniment, paste, tonic, unguent, spray, soap, shampoo, or lip balm containing the active components suspended or dissolved in one or more pharmaceutically acceptable carriers suitable for use in such compositions.

Pharmaceutical compositions of the invention may also be administered in the form of suppositories for rectal administration. These compositions can be prepared by mixing the agents described herein (e.g., one or more of taurine or an analog or derivative thereof (e.g., NCT), dimethylglycine, trimethylglycine, rifamycin antibiotic, macrolide antibiotic, or tetracycline antibiotic) with a suitable non-irritating excipient that is solid at room temperature but liquid at the rectal temperature and therefore will melt in the rectum to release the active components. Such materials include, but are not limited to, cocoa butter, beeswax, and polyethylene glycols.

Pharmaceutical compositions may also be prepared in microcapsules, such as hydroxylmethylcellulose or gelatin-microcapsule and poly-(methylmethacrylate) microcapsule. Pharmaceutical compositions containing agents(s) or a combination thereof (e.g., one or more of taurine or an analog or derivative thereof (e.g., NCT), dimethylglycine, trimethylglycine, rifamycin antibiotic, macrolide antibiotic, or tetracycline antibiotic) may also be prepared in other drug delivery systems such as liposomes, albumin microspheres, microemulsions, nano-particles, and nanocapsules. Such techniques are described in the art. The pharmaceutical compositions to be used for in vivo administration must be sterile. This is readily accomplished by filtration through sterile filtration membranes.

### Combination Therapy

The combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) can be used as a monotherapy (e.g., the combination of anti-chlamydial agents can be administered alone for treating inflammatory diseases associated with or resulting from chlamydial infection) or a combination therapy (e.g., the combination of anti-chlamydial agents can be administered with one or more additional agents (e.g., immunosuppressive drugs) for treating inflammatory diseases associated with or resulting from chlamydial infection). In some embodiments, the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) can be administered to a subject (e.g., a human subject or an animal model) as a combination therapy, e.g., along with one or more immunosuppressive drugs. Immunosuppressive drugs that can be administered to a subject (e.g., a human subject or an animal model) as a combination therapy with the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) may include, but are not limited to, one or more of a corticosteroid, a janus kinase inhibitor, a calcineurin inhibitor, an mTOR inhibitor, an IMDH inhibitor, a biologic, or a monoclonal antibody.

In some embodiments, the immunosuppressive drugs that can be administered to a subject (e.g., a human subject or an animal model) as a combination therapy with the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) is a corticosteroid, such as prednisone (DELTASONE®, ORASONE®), budesonide (ENTOCORT EC®), or prednisolone (MILLIPRED®).

In some embodiments, the immunosuppressive drugs that can be administered to a subject (e.g., a human subject or an animal model) as a combination therapy with the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) is a janus kinase inhibitor, such as tofacitinib (XELJANZ®).

In some embodiments, the immunosuppressive drugs that can be administered to a subject (e.g., a human subject or an animal model) as a combination therapy with the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) is a calcineurin inhibitor, such as cyclosporine (NEORAL®, SANDIMMUNE®, SANGCYA®), or tacrolimus (ASTAGRAF XL®, ENVARSUS XR®, PROGRAF®).

In some embodiments, the immunosuppressive drugs that can be administered to a subject (e.g., a human subject or an animal model) as a combination therapy with the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) is an mTOR inhibitor, such as sirolimus (RAPAMUNE®), or everolimus (AFINITOR®, ZORTRESS®).

In some embodiments, the immunosuppressive drugs that can be administered to a subject (e.g., a human subject or an animal model) as a combination therapy with the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) is an IMDH inhibitor, such as azathioprine (AZASAN®, IMURAN®), leflunomide (ARAVA®), or mycophenolate (CELLCEPT®, MYFORTIC®).

In some embodiments, the immunosuppressive drugs that can be administered to a subject (e.g., a human subject or an animal model) as a combination therapy with the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) is a biologic, such as abatacept (ORENCIA®), adalimumab (HUMIRA®), anakinra (KINERET®), certolizumab (CIMZIA®), etanercept (ENBREL®), golimumab (SIMPONI®), infliximab (REMICADE®), ixekizumab (TALTZ®), natalizumab (TYSABRI®), rituximab (RITUXAN®), secukinumab (COSENTYX®), tocilizumab (ACTEMRA®), ustekinumab (STELARA®), or vedolizumab (ENTYVIO®).

In some embodiments, the immunosuppressive drugs that can be administered to a subject (e.g., a human subject or an animal model) as a combination therapy with the combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) is a monoclonal antibody, such as basiliximab (SIMULECT®), or daclizumab (ZINBRYTA®).

### Method of Treatment

Methods of treatment, dosage levels and requirements featured herein may be selected by those of ordinary skill in the art from available methods and techniques.

It will be appreciated that the actual dosages of the agents (taurine, dimethylglycine, trimethylglycine, rifamycin antibiotic, macrolide antibiotic, or tetracycline antibiotic) or pharmaceutical compositions containing the agents as a monotherapy or a combination therapy will vary according to the particular composition formulated, the mode of administration, the particular disease being treated, and the particular subject being treated. Those skilled in the art using conventional dosage-determination tests in view of the experimental data may ascertain optimal dosages for a given set of conditions. For oral administration, an exemplary dose of the agents (e.g., anti-chlamydial agents) will be: about 1000 mg taurine (e.g., about 100, 120, 130, 140, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, or 5000 mg taurine); about 1200 mg dimethylglycine (e.g., about 100, 120, 130, 140, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, or 5000 mg dimethylglycine); about 1500 mg trimethylglycine (e.g., about 100, 120, 130, 140, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, or 5000 mg trimethylglycine); about 150 mg of a rifamycin antibiotic, such as Rifabutin (e.g., about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 mg, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mg of a rifamycin antibiotic, such as Rifabutin); about 250 mg of a macrolide antibiotic, such as Azithromycin (e.g., about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 mg, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mg of a macrolide antibiotic, such as Azithromycin); and/or about 100 mg of a tetracycline antibiotic, such as Minocycline (e.g., about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 mg, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mg of a tetracycline antibiotic, such as Minocycline).

The course of treatment can be repeated (e.g., 1-12 times or more (e.g., 1 time, 2 times, 3 times, 4 times, 5 times, 6 times, 7 times, 8 times, 9 times, 10 times, 11 times, 12 times, or more)) at appropriate intervals, e.g., every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 1 year. The combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) may be administered to a subject in need thereof, for example, one or more times (e.g., 1-12 times or more) hourly, daily, weekly, biweekly, monthly, bimonthly, quarterly, biannually, annually, or as medically necessary. Dosages may be provided in either a single or multiple dosage regimens. The timing between administrations may decrease as the medical condition improves or increase as the health of the patient declines. The treatment may be continued for 5 years or less (e.g., 5 years, 4 years, 3 years, 2 years, 1 year, 6 months, 5 months, 4 months, 3 months, 8 weeks, 7 weeks, 6 weeks, 5 weeks, 4 weeks, 3 weeks, 15 days, 14 days, 13 days, 12 days, 11 days, 10 days, 9 days, 8 days, 7 days, 6 days, 5 days, 4 days, 3 days, 2 days, or less). For example, an inflammatory disease (e.g., inflammatory disease associated with or resulting from chlamydial infection) in a subject may be treated by administering (e.g., orally) to the subject a combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) as a monotherapy (e.g., the combination of anti-chlamydial agents alone) or a combination therapy (e.g., the combination of anti-chlamydial agents with one or more immunosuppressive drugs (e.g., one or more corticosteroids, janus kinase inhibitors, calcineurin inhibitors, mTOR inhibitors, IMDH inhibitors, biologics, or monoclonal antibodies)) for 5 years or less (e.g., 5 years, 4 years, 3 years, 2 years, 1 year, 6 months, 5 months, 4 months, 3 months, 8 weeks, 7 weeks, 6 weeks, 5 weeks, 4 weeks, 3 weeks, 15 days, 14 days, 13 days, 12 days, 11 days, 10 days, 9 days, 8 days, 7 days, 6 days, 5 days, 4 days, 3 days, 2 days, or less).

The method of treatment described herein may include administration (e.g., oral administration) of each of the agents (e.g., each of the anti-chlamydial agents) separately, simultaneously (e.g., administration of the agents at the same time) or sequentially (e.g., administration of the agents within 24 hours or less (e.g., within 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5.5 hours, 5 hours, 4.5 hours, 4 hours, 3.5 hours, 3 hours, 2.5 hours, 2 hours, 1.5 hours, 1 hour, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, 1 minute, or less) of each other) over the course of therapy. For example, at least one agent effective against (e.g., that effectively reduces or eliminates) the EB phase of chlamydial life cycle (e.g., one or more of taurine or an analog or derivative thereof (e.g., NCT), dimethylglycine, and trimethylglycine) can be administered (e.g., orally) simultaneously (e.g., at the same time) with at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic). Alternatively, at least one agent effective against (e.g., that effectively reduces or eliminates) the EB phase of chlamydial life cycle (e.g., one or more of taurine or an analog or derivative thereof (e.g., NCT), dimethylglycine, and trimethylglycine) can be administered (e.g., orally) within 24 hours (e.g., within 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5.5 hours, 5 hours, 4.5 hours, 4 hours, 3.5 hours, 3 hours, 2.5 hours, 2 hours, 1.5 hours, 1 hour, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, 1 minute, or less) of administering at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic).

In some embodiments, the method of treatment may further include a diagnostic method, such as a test or assay (e.g., test or assay including polymerase chain reactions, ELISA, luminescence assays (e.g., fluorescence and chemiluminescence), radioimmunoassay, and/or immunohistology) to detect the presence and/or amount of chlamydial nucleic acids or proteins in a subject (e.g., in a biological material (e.g., bodily secretion, bodily fluid, and/or tissue) from the subject) who is receiving the treatment. The duration of treatment regimen may depend on the outcome of the diagnostic method. In particular, treatment of a subject with a combination of agents (e.g., anti-chlamydial agents) described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic)) as a monotherapy (e.g., the combination of anti-chlamydial agents alone) or a combination therapy (e.g., the combination of anti-chlamydial agents with one or more immunosuppressive drugs) may be concluded when results of the diagnostic method (e.g., test or assay to detect the presence and/or amount of chlamydial nucleic acids or proteins in the subject) indicate reduction or elimination of chlamydial infection and/or the different phases of chlamydial life cycle (e.g., the EB phase of chlamydial life cycle, the cryptic phase of chlamydial life cycle, and the replicating phase of chlamydial life cycle) in the subject (e.g., in a biological material from the subject). For example, treatment of a subject with a combination of agents described herein may be concluded when results of the diagnostic method (e.g., test or assay to detect the presence and/or amount of chlamydial nucleic acids or proteins in the subject) indicate reduction of chlamydial infection and/or reduction of the different phases of chlamydial life cycle (e.g., the EB phase of chlamydial life cycle, the cryptic phase of chlamydial life cycle, and the replicating phase of chlamydial life cycle) in the subject (e.g., in a biological material from the subject) by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)). Additionally, treatment of a subject with a combination of agents described herein may be concluded when results of the diagnostic method (e.g., test or assay to detect the presence and/or amount of chlamydial nucleic acids or proteins in the subject) indicate elimination of chlamydial infection and/or elimination of the different phases of chlamydial life cycle (e.g., the EB phase of chlamydial life cycle, the cryptic phase of chlamydial life cycle, and the replicating phase of chlamydial life cycle) in the subject (e.g., in a biological material from the subject), such as when the subject (e.g., biological material from the subject) tests negative for chlamydial infection by the diagnostic method.

Upon improvement of a subject's condition, upon reduction or elimination of chlamydial infection in the subject (e.g., in a biological material in the subject), and/or upon elimination of the different phases of chlamydial life cycle in the subject (e.g., in a biological material in the subject), a maintenance dose of a composition of this invention may be administered if necessary. Subsequently, the dosage or frequency of administration, or both, may be reduced, as a function of the symptoms, to a level at which the improved condition is retained. When the symptoms have been reduced or alleviated to the desired level, treatment should cease, at least in principle. Patients may, however, require intermittent treatment on a long-term basis, upon any recurrence of the inflammatory disease.

As the skilled artisan will appreciate, lower or higher doses than those recited above may be required. Specific dosage and treatment regimen for any particular patient will depend upon a variety of factors, including the activity of the specific composition used, the age, body weight, general health status, sex, diet, time of administration, rate of excretion, the severity of the disease, the patient's disposition to the disease and the judgment of the treating physician.

With respect to the composition of the present invention, the particular pharmaceutical formulation, the dosage, and the number of doses given per day to a mammal (e.g., a human or an animal model) requiring such treatment are all choices within the knowledge of one of ordinary skill in the art and can be determined without undue experimentation.

### EXAMPLES

The following examples are put forth to provide those of ordinary skill in the art with a description of how the combination of agents described herein may be used, and how the methods featured herein may be evaluated. The examples are intended to be purely exemplary of the invention and are not intended to limit the scope of the claims.

### Example 1

### Treatment of an Inflammatory Disease Associated with Chlamydial Infection

The methods and compositions described herein (e.g., a combination of: (a) at least one agent effective against (e.g., that effectively reduces or eliminates) the EB phase of chlamydial life cycle (e.g., one or more of taurine or an analog or derivative thereof (e.g., NCT), dimethylglycine, and trimethylglycine), and (b) at least one agent effective against (e.g., that effectively reduces or eliminates) the cryptic phase and/or replicating phase of chlamydial life cycle (e.g., one or more of a rifamycin antibiotic, a macrolide antibiotic, and a tetracycline antibiotic); and method of treatment therewith) are useful for treating inflammatory diseases that are associated with or result from chlamydial infection. Combination of agents described herein can reduce or eliminate chlamydial infection in a subject (e.g., a human) by reducing or eliminating the different phases of chlamydial life cycle (e.g., the EB phase of chlamydial life cycle, the cryptic phase of chlamydial life cycle, and the replicating phase of chlamydial life cycle), thereby treating an inflammatory disease in the subject that is associated with or results from the chlamydial infection. For instance, a subject with chlamydial infection and one or more inflammatory diseases resulting therefrom can be treated (e.g., orally) with a combination of: (a) one or more of about 1000 mg taurine, about 1200 mg dimethylglycine, and about 1500 mg trimethylglycine; and (b) one or more of about 150 mg of a rifamycin antibiotic (e.g., Rifabutin), about 250 mg of a macrolide antibiotic (e.g., Azithromycin), and about 100 mg of a tetracycline antibiotic (e.g., Minocycline) to treat the chlamydial infection and the inflammatory disease resulting therefrom. Administration of the combination of anti-chlamydial agents, for instance, is useful for reducing or eliminating chlamydial infection and the different phases of chlamydial life cycle in the subject. Reduction or elimination of chlamydial infection and the different phases of chlamydial life cycle in the subject can be determined by a diagnostic method that detects the presence or amount of chlamydial nucleic acid and protein in a biological material from the subject. Administration of the combination of anti-chlamydial agents, for instance, is also useful for reducing inflammation in the subject and/or reducing one or more symptoms of the inflammatory disease in the subject by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)). A single dose or more than one dose of the combination of agents (e.g., anti-chlamydial agents) described herein or a composition containing the same is administered to the subject over a course of days, weeks, months, or years. The treatment is continued until the subject (e.g., a biological material from the subject) tests negative for chlamydial infection by a diagnostic method (e.g., a test or assay that detects chlamydial nucleic acid or protein in the subject (e.g., in the biological material from the subject)), such as when chlamydial nucleic acid or protein is not undetectable in the subject (e.g., in the biological material from the subject) by the diagnostic method. Additionally, the progression of the inflammatory disease is monitored by any one or more of several established methods. A physician typically monitors the subject by direct observation in order to evaluate how the symptoms exhibited by the subject have changed in response to treatment. Based on such observations, a physician may prescribe higher/lower dosages or more/less frequent dosing of the anti-chlamydial agents or a composition containing the same in subsequent rounds of treatment.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

What is claimed is:

1. A method of treating a chronic inflammatory disease in a subject in need thereof, the method comprising administering to the subject at least two agents, wherein each agent is effective against a different phase of chlamydial life cycle and is selected from the group consisting of:
(a) agent effective against elementary body (EB) phase of chlamydial life cycle, wherein the agent is one or more of taurine, dimethylglycine, and trimethylglycine, or an analog or derivative thereof; and
(b) agent effective against cryptic phase and/or replicating phase of chlamydial life cycle, wherein the agent is one or more of a rifamycin antibiotic, a macrolide antibiotic, and a tetracycline antibiotic,
and wherein, one or more agents from group (a) are administered within 24 hours of administering one or more agents from group (b).

2. The method of claim 1, wherein the one or more agents from group (a) are administered within 12 hours or 6 hours of administering the one or more agents from group (b).

3. (canceled)

4. The method of claim 1, wherein the one or more agents from group (a) comprises taurine; a combination of taurine and dimethylglycine; or a combination of taurine, dimethylglycine and trimethylglycine.

5. The method of claim 1, wherein the taurine analog is N-chlorotaurine (NCT).

6. The method of claim 5, wherein the NCT is in a 1% (w/v) aqueous solution

7. The method of claim 1, wherein the one or more agents from group (b) comprises a rifamycin antibiotic, a macrolide antibiotic, and a tetracycline antibiotic.

8. The method of claim 1, wherein the rifamycin antibiotic is selected from the group consisting of rifabutin, rifampicin, and rifapentin.

9. (canceled)

10. The method of claim 1, wherein the macrolide antibiotic is selected from the group consisting of azithromycin, erythromycin, clarithromycin, roxithromycin, spiramycin, oleandomycin, josamycin, kitsamysin, and flurithromycin.

11. (canceled)

12. The method of claim 1, wherein the tetracycline antibiotic is selected from the group consisting of minocycline, doxycycline, chlortetracycline, tetracycline hydrochloride, oxytetracycline, demeclocycline, and methacycline.

13. (canceled)

14. The method of claim 1, wherein:
(a) the inflammatory disease is caused by chlamydial infection in the subject;
(b) the method reduces or eliminates chlamydial infection; and/or
(c) the method reduces one or more symptoms of the inflammatory disease in the subject.

15. (canceled)

16. The method of claim 1, further comprising performing a test, wherein the test detects EB phase of chlamydial life cycle, replicating phase of chlamydial life cycle, and cryptic phase of chlamydial life cycle in a biological material from the subject.

17. The method of claim 1, wherein the agents are administered to the subject until the biological material from the subject is negative for Chlamydia according to the test.

18. The method of claim 16, wherein the biological material comprises bodily secretion, bodily fluid, and/or tissue from the subject and/or wherein the test is an assay for detection of chlamydial nucleic acid or protein.

19. (canceled)

20. The method of claim 18, wherein the assay comprises polymerase chain reaction, enzyme-linked immunosorbent assay (ELISA), radioimmunoassay, and/or immunohistology.

21. (canceled)

22. The method of claim 1, wherein the inflammatory disease is a respiratory disease, a neurodegenerative disease, a musculoskeletal disease, or a cardiovascular disease.

23. The method of claim 22, wherein:
(a) the respiratory disease is a respiratory condition involving inflammation or infection of a respiratory mucosa; a respiratory condition involving inflammation or infection of an underlying muscle of the respiratory tract; cystic fibrosis; pneumonia; asthma; bronchitis; sinusitis or rhinosinusitis; infection of a sinus; chronic obstructive airway disease; emphysema; chronic bronchitis; or bronchiectasis;
(b) the neurodegenerative disease is Alzheimer's disease, Parkinson's disease, Prion disease, motor neuron diseases, Huntington's disease, spinocerebellar ataxia, or spinal muscular atrophy;
(c) the musculoskeletal disease is arthritis, tendinitis, carpal tunnel syndrome, or fibromyalgia; or
(d) the cardiovascular disease is atherosclerosis, coronary artery diseases, stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, or venous thrombosis.

24-30. (canceled)

31. The method of claim 1, further comprising administering to the subject one or more immunosuppressive drugs.

32. The method of claim 31, wherein the immunosuppressive drug is one or more of a corticosteroid, a janus kinase inhibitor, a calcineurin inhibitor, an mTOR inhibitor, an IMDH inhibitor, a biologic, or a monoclonal antibody.

33. The method of claim 32, wherein:
(i) the corticosteroid is prednisone, budesonide, or prednisolone;
(ii) the janus kinase inhibitor is tofacitinib;
(iii) the calcineurin inhibitor is cyclosporine or tacrolimus;
(iv) the mTOR inhibitor is sirolimus or everolimus;
(v) IMDH inhibitor is azathioprine, leflunomide, or mycophenolate;
(vi) the biologic is abatacept, adalimumab, anakinra, certolizumab, etanercept, golimumab, infliximab, ixekizumab, natalizumab, rituximab, secukinumab, tocilizumab, ustekinumab, or vedolizumab; and/or
(vii) the monoclonal antibody is basiliximab or daclizumab.

34. The method of claim 1, wherein the subject is a human.

35-64. (canceled)
